(19) United States
(12) Patent Application Publication
(10) Pub. No.: US 2017/0291021 A1
(43) Pub. Date: Oct. 12, 2017

(54) Peritoneal Conduit

(71) Applicant: Nouzhan Sehati, Los Angeles, CA (US)

(72) Inventor: Nouzhan Sehati, Los Angeles, CA (US)

(21) Appl. No.: 15/095,543

(22) Filed: Apr. 11, 2016

(51) Int. Cl. A61M 39/02; A61M 27/00; A61B 17/34; A61M 25/04

(52) U.S. Cl. CPC A61M 39/0247; A61M 25/04; A61M 27/006; A61B 17/3421; A61M 2025/0286

(57) ABSTRACT

The Peritoneal Conduit is a medical device composed of tubing with an integrated collar made of a flexible and non-absorbable material for surgical implantation to create a conduit for passage of a catheter between two body cavities. The Peritoneal Conduit has a hollow center that spans from one end (shorter proximal portion) to the other end (longer distal portion). The catheter that will be being placed through the Peritoneal Conduit will enter into the proximal portion and will pass through the hollow center of the Peritoneal Conduit to emerge from the distal portion. The catheter is then secured to the Peritoneal Conduit using one suture that is tied around the groove in the proximal portion of the Peritoneal Conduit. The Peritoneal Conduit itself is secured in placed using two sutures that are placed through the integrated collar at the junction of the proximal and distal portions of the Peritoneal Conduit.

### CROSS-REFERENCE TO RELATED APPLICATIONS

This application claims the benefit of U.S. Provisional Application No. 62/157,468, filed on May 5, 2015, the disclosure of which is incorporated herein by reference.

### STATEMENT REGARDING FEDERALLY SPONSORED RESEARCH OR DEVELOPMENT

Not applicable.

### BACKGROUND OF THE INVENTION

Cerebrospinal fluid (CSF) is a clear and colorless fluid that is constantly produced and reabsorbed by the central nervous system, and circulates inside of various spaces that are inside and around the brain and spinal cord. A variety of diseases can lead to abnormal accumulation of CSF within the nervous system which can lead to disabling symptoms and may even be fatal. Such conditions are remedied by surgical placement of a CSF diversion shunt, a variety of which exist depending on the exact nature of the disease (e.g. ventriculoperitoneal shunt, cyst-peritoneal shunt, lumboperitoneal shunt, syrinx-peritoneal shunt, etc.). During placement of a CSF diversion shunt the tip of a short, thin, and flexible catheter is placed inside of the cavity that contains the abnormal CSF collection (e.g. cerebral ventricles, arachnoid cyst, subarachnoid space, syrinx within the spinal cord, etc.). The other end of this catheter is typically attached to a valve which regulates the amount of CSF that is drained by the shunt system; and the valve is then connected to a long, thin, and flexible tube called the peritoneal catheter. This tube is then tunneled under the skin toward the cavity in which the distal end of the catheter will be placed (i.e. the abdominal cavity). The excess CSF will then flow through this shunt system and drain into the abdominal cavity which then allows for the absorption of the diverted CSF into the blood stream.

In order to surgically place the distal end of the peritoneal catheter inside of the abdominal cavity, the surgeon makes an incision in the skin over the abdomen (typically about 3-5 cm in length); and dissects the layers of tissue under the skin until the superficial fascia layer is reached. A small linear opening is then made in this layer, and the underlying muscle layer is dissected until the deep fascia layer is visualized. A very small opening is then made in the deep fascia layer and in the underlying layer (called peritoneum), which gives the surgeon access into the abdominal cavity. The distal end of the peritoneal catheter, is then tunneled under the skin from its attachment to the valve, through the subcutaneous tissues, until it reaches the abdominal incision. The distal tip is then passed into the abdominal cavity through the opening into the abdominal cavity; and typically about 20 to 50 cm of tubing is passed into the abdominal cavity based on surgeon discretion. After the tubing is placed inside of the abdominal cavity, the various layers which were previously opened are sutured closed around the catheter. This implanted shunt systems will then continuously drain the excess CSF collection into the abdominal cavity, and thereby relieve the associated symptoms that were caused by the accumulation of the excess CSF.

One challenge for the surgeon during the placement of these peritoneal catheters is that the material from which most of these catheters are made causes the surface of the catheters to be somewhat slippery with a tendency to slide easily across biological tissues. This obligates the surgeon to secure the peritoneal catheter to the surrounding tissues (after it has been placed inside of the abdominal cavity) in order to prevent the tip of the catheter from backing out of the abdominal cavity, which would require surgical replacement. Securing the catheter is often done by tying a suture around the catheter and fastening the suture to the local tissue. The concern for the surgeon is that if the suture is tied too tightly around the thin and flexible catheter, then it can potentially partially or completely occlude the lumen of the catheter and lead to suboptimal drainage of CSF. If the suture is tied too loosely around the catheter, then the tubing is at an increased risk of backing out as explained above. Therefore, determining the appropriate amount of tension to be applied to the securing suture around the catheter can sometimes be somewhat challenging, especially to the less experienced surgeon. In addition, even if the tubing is secured properly at the time of surgery, it can still potentially loosen at a later time, allowing the peritoneal catheter to back out of the abdominal cavity.

Another issue regarding these CSF diversion systems stems from their tendency to need revision over time. These shunt systems are often placed with the intention for them to stay inside the patient's body for the remainder of their lives because the medical condition that typically leads to the placement of these shunts tends to be a pervasive and often a life-long problem. Despite improvements in design, however, a relatively significant number of these shunts fail over time, and the entire system or one its components needs to be replaced at some point during the patient's lifetime due to malfunction or obstruction. A considerable number of patients may even need multiple surgeries for repeated malfunction of their shunts over their lifespan. One of the components of the shunt system that is typically more prone to blockage is the long peritoneal catheter. This portion is usually repaired by removing the existing (malfunctioning) catheter first and then placing another peritoneal catheter by repeating the surgical procedure for placement of the peritoneal catheter that was described above.

With each subsequent surgery the risks of complications for the patient increases. One reason for this is that with repeat operations the surgeon has to dissect through layers of scar which were left by the previous surgeries in order to gain access into the abdominal cavity; and this is more difficult to do and riskier to the patient with each subsequent surgery. This risk can potentially be circumvented by making a new incision in another part of the abdomen through normal (non-scarred) tissue; however, this options is esthetically displeasing to patients since it leaves yet another unsightly scar. In addition, repeated entry into the abdominal cavity can increase the risk of damage to the bowel and may cause scarring of the abdominal contents which can lead to patient morbidity over time, and can make subsequent surgeries for shunt malfunction more prone to bowel injury and technically more challenging.

### BRIEF SUMMARY OF THE INVENTION

The invention that is the subject of this inquiry, called the Peritoneal Conduit (PC), was designed to address the issues mentioned above as well as any other similar issues that may be addressed through the proper use of this device.

The design of the PC is described in detail in the following section titled “DETAILED DESCRIPTION OF THE INVENTION.”

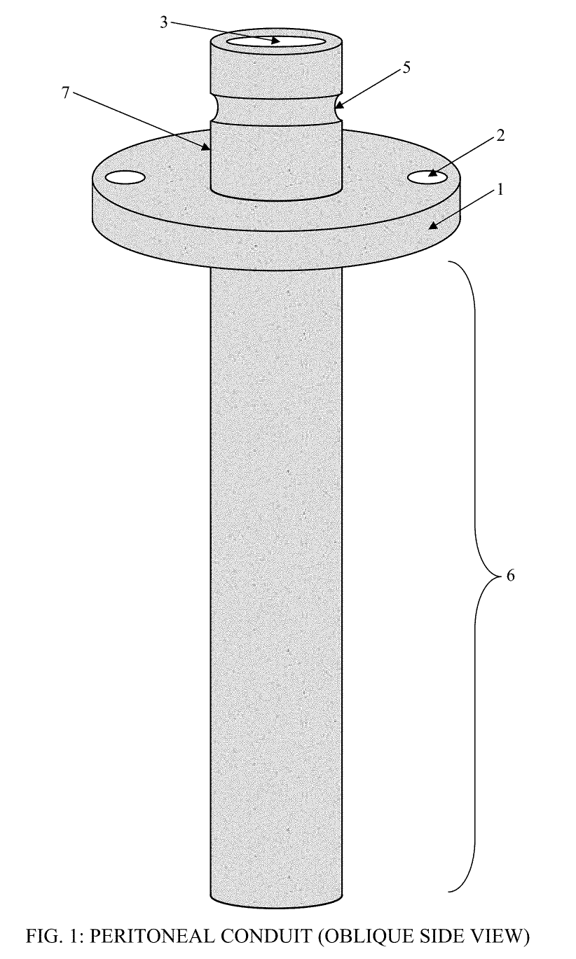

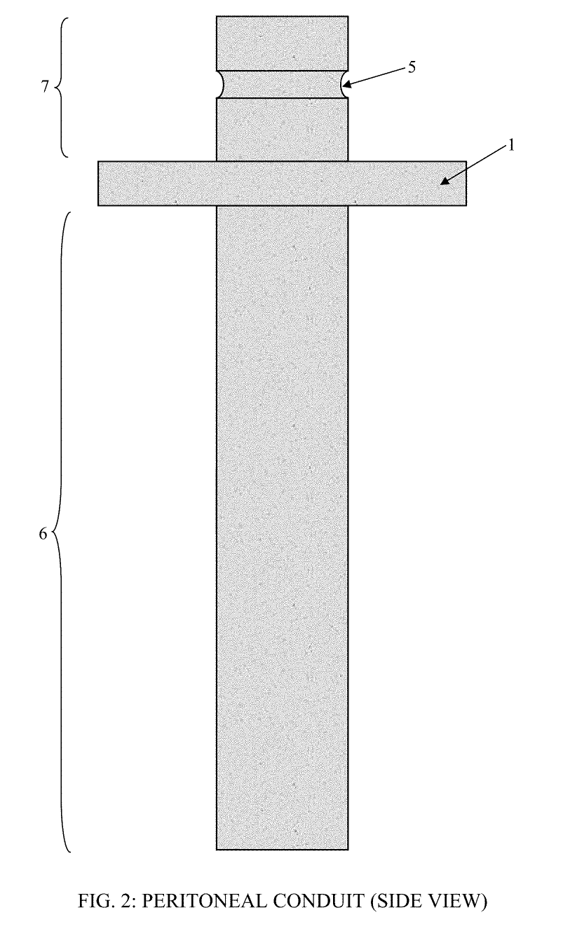

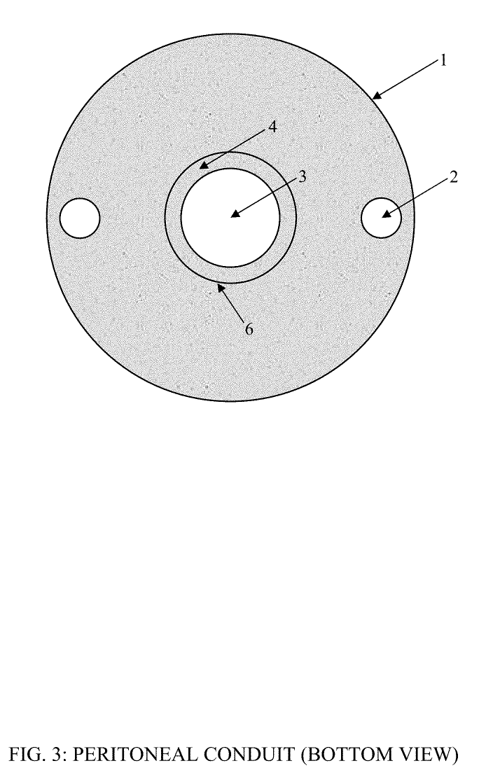

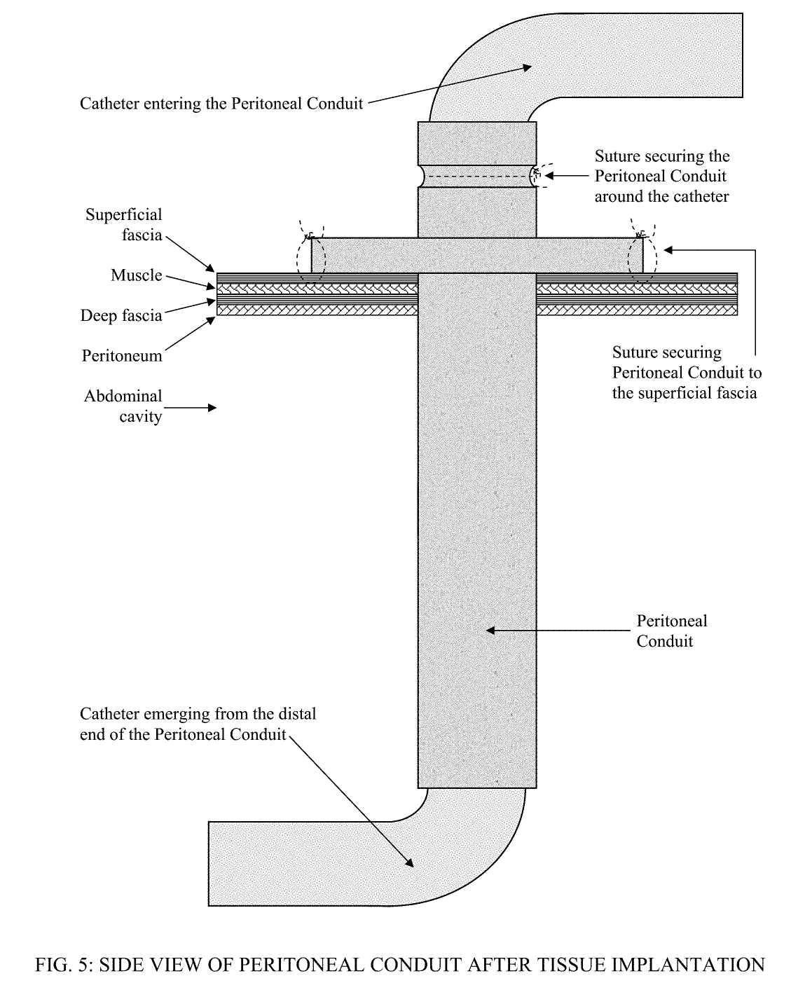

The surgical implantation of the Peritoneal Conduit does not require any special surgical skill sets other than those that are needed for placement of a CSF diversion shunt using standard surgical techniques. In that regard, after the surgeon gains access into the abdominal cavity during the placement of a CSF diversion shunt (as described above), the distal portion of the PC (Component #6 in FIGS. 1, 2 & 3) is passed into the abdominal cavity through the small opening surgically made into this cavity (as described above) until the underside of the integrated collar (Component #1 in FIGS. 1, 2, 3 & 4) comes to rest on the superficial fascia layer (FIG. 5). The integrated collar of the PC remains outside of the superficial fascia layer, and after this fascia layer is closed in the standard manner, the collar is secured to this layer using non-absorbable suture (FIG. 5) which is placed through the two suture holes (Component #2 in FIGS. 1, 3 & 4) in the integrated collar of the PC. The peritoneal catheter is then passed through the hollow center of the PC (Component #3 in FIGS. 1, 3 & 4) and into the abdominal cavity. The peritoneal catheter is then secured in place by tying a non-absorbable suture around the groove in the proximal portion of the PC (Component #5 in FIGS. 1 & 2) which will secure the peritoneal catheter inside of the PC through friction between the inside wall of the PC and the outside wall of the peritoneal catheter. The remainder of the wound is then closed in a standard manner. FIG. 5 depicts the side view of the final configuration of the PC in the tissue at the conclusion of the procedure.

If replacement of the peritoneal catheter is needed at a later time, then the securing suture in the groove in the proximal portion of the PC may be easily removed and the existing peritoneal catheter can be taken out through the PC. A new peritoneal catheter can then be replaced through the PC without the need to create another opening into the abdominal cavity. Furthermore, this allows for a significantly smaller incision to be made because only the peritoneal catheter and the proximal portion of the PC (Component #7 in FIGS. 1, 2 & 4) needs to be dissected in order to complete the abdominal portion of the peritoneal catheter replacement.

The description above is meant as an illustration of the main intended utility of the Peritoneal Conduit. However, this invention can be used for surgical implantation into any tissue in order to create a permanent conduit from one anatomic space into another anatomic space across biological barrier(s).

### DETAILED DESCRIPTION OF THE INVENTION

The PC is a catheter with an integrated collar (Component #1 in FIGS. 1, 2, 3 & 4) that is made of a flexible and non-absorbable material for the purpose of surgical implantation in any tissue in order to create a permanent conduit from one anatomic space into another anatomic space across biological barriers.

The tubing can be made of any surgically-implantable flexible and non-absorbable material such as silicone/Silastic® and can be barium impregnated to allow for better identification using x-ray imaging. The PC can also be antibiotic impregnated in order to reduce the likelihood of infection in patients at higher risk of infections. Impregnation with barium and/or antibiotics can be done through methods that are currently in wide use in the medical industry.

The length of the distal portion of the PC (Component #6 in FIGS. 1, 2 & 3) and the proximal portion of the PC (Component #7 in FIGS. 1, 2 & 4) is variable and both of these components can be trimmed at the surgeon's discretion in order to custom-fit the PC to the anatomy of the patient. The inside diameter of the tubing (Component #3 in FIGS. 1, 3 & 4) can also be manufactured in various sizes to accommodate for the difference in the sizes of the potential catheters that may need to be placed through the conduit.

The PC can also be manufactured with different thicknesses of the wall of the tubing (Component #4 in FIGS. 3 & 4) to allow for variability in different biological tissues and for surgeon preference, with the understanding that thicker tubing wall will likely lead to less flexibility of the tubing, and thinner tubing wall may allow for more flexibility depending on which is desired based on the patient's anatomic needs.

### BRIEF DESCRIPTION OF THE DRAWINGS

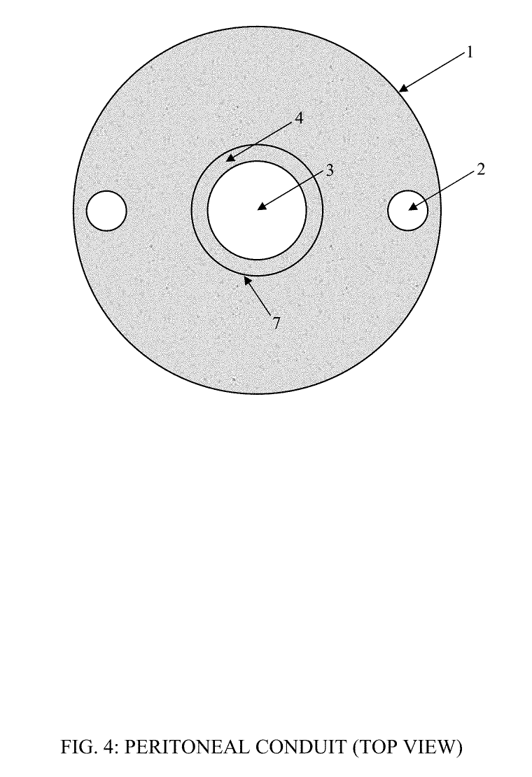

There are 5 figures that are included with this application. The images are not drawn to scale and are intended to depict the general appearance and the salient features of the Peritoneal Conduit (PC). FIG. 1 is a schematic drawing showing the oblique side view of the PC. FIG. 2 is a schematic drawing showing the side view of the PC. FIG. 3 is a schematic drawing showing the bottom view of the PC. FIG. 4 is a schematic drawing showing the top view of the PC. FIG. 5 is a schematic drawing showing the side view of the Peritoneal Conduit after implantation in tissue (the example used here is the abdominal tissue with some of its layers such as superficial fascia, muscle, deep facia, peritoneum, and the abdominal cavity).

The description of the numbered components in the above drawings are as follows: 1=Integrated collar, 2=Suture hole, 3=Hole running through the center of the conduit, 4=Cross section of the wall of the conduit, 5=Groove for securing suture in the proximal portion of the conduit, 6=Distal portion of the conduit, and 7=Proximal portion of the conduit.

1. A Peritoneal Conduit comprising: a tubing having a first and a second end; and an integrated collar; wherein the said integrated collar is near one end of the said tubing.

2. The invention of claim 1 wherein the tubing and integrated collar are made of a flexible and non-absorbable material such as silicone/Silastic®.

3. The invention of claim 1 wherein there is a thin groove in the outside wall of the tubing between the integrated collar and the first end.

4. The invention of claim 1 wherein the tubing has a hollow center throughout its entire length that spans the tubing from its first end to the second end.

5. The invention of claim 1 wherein the tubing may be trimmed at the first end and/or the second end in order to fit the biological tissue in which it is implanted.

6. The invention of claim 1 wherein the integrated collar may be of any shape, size, or thickness;
and it exists for the purpose of securing the invention to surrounding tissues after implantation.

7. The invention of claim 1 wherein the device is used for surgical implantation in order to create a permanent conduit through the said hollow center from one anatomic space into another anatomic space across biological barriers.
